(19) United States
(12) Plant Patent Application Publication
(10) Pub. No.: US 2002/0073459 P1
(43) Pub. Date: Jun. 13, 2002

(54) Chrysanthemum named medea

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/734,657

(22) Filed: Dec. 13, 2000

(51) Int. Cl. A01H 5/00

(52) U.S. Cl. PLT/296

(57) ABSTRACT

A chrysanthemum plant named MEDEA characterized by its small sized blooms with apricot ray florets and prolific branching; natural season flower date August 21-27; blooming for a period of 4 weeks.

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from the open pollination among groups of chrysanthemum cultivars maintained under the control of the inventor for breeding purposes. The new and distinct cultivar was discovered and selected as one flowering plant by Rob Noodelijk on a cultivated field in Rijsenhout Holland in September 1998. The plant has been asexually reproduced by cuttings in greenhouses at Rijsenhout Holland. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in fall bloom.

FIG. 2 shows the various stages of bloom and foliage of the new cultivar.

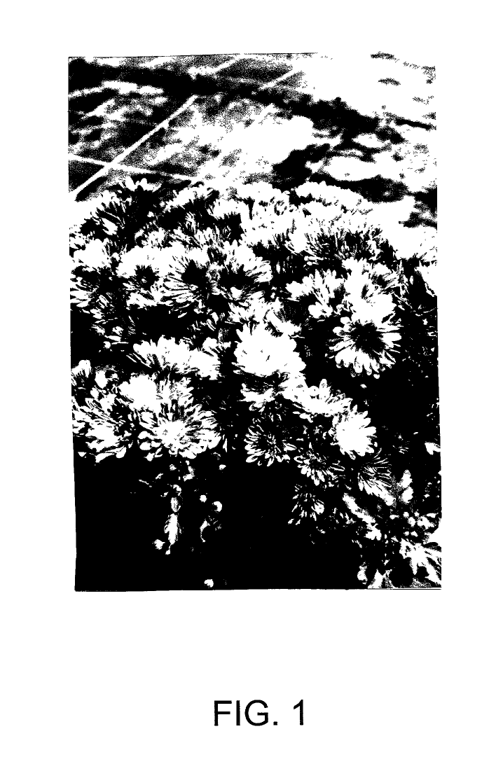

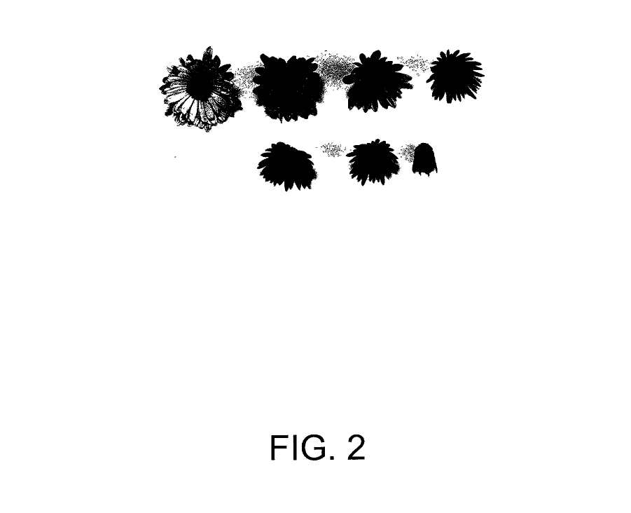

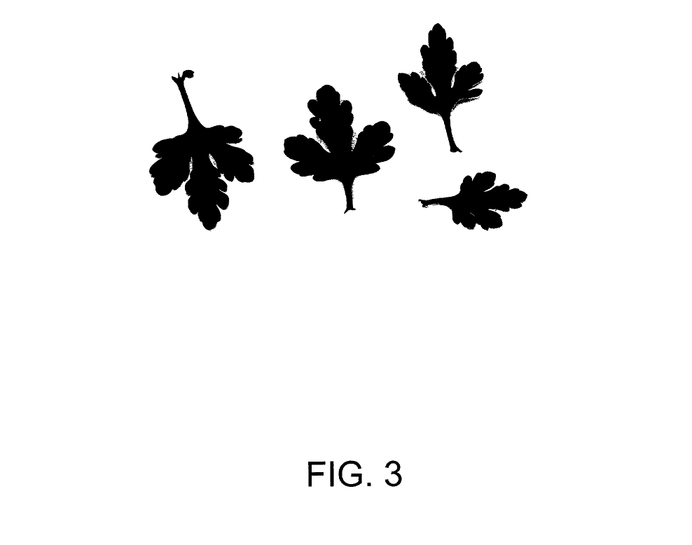

### DESCRIPTION OF THE INVENTION

This new variety of chrysanthemum is of the botanical classification Dendranthema Grandiflora. The observations and measurements were gathered from plants grown out door in Rijsenhout, Holland under natural day length and temperature and planted week 22 in 1999 and 2000. The natural blooming date of this crop was August 21-27 (week 34). The average height of the plants was 36-40 cms. This new variety produces small sized blooms with apricot ray florets and yellow-orange disc-florets blooming for a period of 4 weeks.

The following is a description of the plant and characteristics that distinguish MEDEA as a new and distinct variety.

The color designations are taken from the plant itself. Accordingly, any discrepancies between the color designations and the colors depicted in the photographs are due to photographic tolerances.

Botanical Classification: Cultivar Medea.

Bud.—

Size.—Small, cross-section +/−1.0 cm height +/−1.1 cm

Outside Color.—Greyed-orange 176A

Bloom.—

Size.—Small

Fully expanded.—4.0 cms

Borne (number of blooms per branch).—Upper and lower portion plural blooms per branch (approx. 7 blooms per branch)

Length of lateral branch.—From top till bottom 17-19 cm

Lateral branch, Attachment.—Strong

Branching.—Prolific with 9-11 breaks after pinching

Form.—Daisy

Performance (on the plant).—4 weeks

Color.—

Center of flower.—Immature yellow-green 153A Mature yellow-orange 14A

Color of the upper surface of the majority of the ray-florets.—Greyed-orange 165D

Color of the lower surface of the majority of the ray-florets.—Greyed-orange 166D

Tonality from distance.—A daisy garden mum with apricot blooms

Discoloration.—To greyed-orange 164D

Ray florets.—

Texture.—Upper side and under side smooth

Number.—55-60

Cross-section.—Flat sometimes concave

Longitudinal axis of the majority.—Straight sometimes incurved

Shape of tip.—Rounded

Fragrance.—Typical chrysanthemum

Cultivar Medea:

Disc Diameter (natural season crop when visible).—0.8 cms

Reproductive Organs.—

Stamen.—Yellow-orange, thin, length +/−2 mm

Pollen.—Present

Styles (present in both my and disc florets).—Green, thin

Style length.—+/−3 mm

Stigmas.—Yellow-green

Stigma width.—+/−1 mm

Ovaries.—Enclosed in calyx

Plant.—

Form.—A garden mum (pot mum) meant for out door use

Growth.—Vigorous

Height (from soil level).—36-40 cms

Stem color.—Green 138B

Stem strength.—Medium

Stem brittleness.—Absent

Stem anthocyanin coloration.—Present

Natural blooming date.—August 21-27

Foliage.—

Color.—Upper side yellow-green 146B Under side yellow-green 146C

Size.—Very small, length +/−4.0 cm width+/−3.5 cm

Quantity (number per lateral branch).—9-11

Shape.—Egg shaped

Texture.—Fleshy

Ribs and Veins.—Ribs and veins not so well developed

Edge.—Crenated

Shape of base of sinus between lateral lobes.—Acute

Margin of sinus between lateral lobes.—Converging

Shape of base.—Truncate sometimes asymmetrical

Cultivar Medea:

Apex.—cuspidate

I claim:

1. A new and distinc variety of chrysanthemum plant as described and illustrated
